(19) United States
(12) Patent Application Publication
(10) Pub. No.: US 2024/0233058 A1
(43) Pub. Date: Jul. 11, 2024

(54) ONLINE MENTOR MENTEE RELATIONSHIP MANAGEMENT SYSTEM

(71) Applicant: Brittany Leshon O'Bryant, Marietta, GA (US)

(72) Inventor: Brittany Leshon O'Bryant, Marietta, GA (US)

(21) Appl. No.: 18/151,748

(22) Filed: Jan. 9, 2023

(51) Int. Cl. G06Q 50/20; G16H 20/70

(52) U.S. Cl. CPC G06Q 50/2057; G16H 20/70

(57) ABSTRACT

A user relationship management system is described. The system may include a transceiver configured to receive a first user profile and a target qualification from a first user device, and a second user profile from a second user device. The system may further include a processor configured to determine that a second user qualification matches with the target qualification. The processor may be further configured to compare the first user profile and the second user profile to calculate a match score between the first user profile and the second user profile, when the second user qualification matches with the target qualification. The processor may further compare the match score with a predefined threshold and transmit the second user profile to the first user device when the match score is greater than the predefined threshold.

### TECHNICAL FIELD

The present disclosure relates to an online system and method for management of mentor-mentee relationship, and more particularly, to a system and method that facilitates mentor and mentee match making based on profiles and preferences of mentor and mentee.

### BACKGROUND

Graduates in mental healthcare programs are required to accrue a predefined count of hours under supervision of a mentor to become fully licensed. For example, a license-eligible graduate in a Clinical Mental Health Counseling program may be required to work under supervision of a certified professional counselor supervisor (CPCS) for 3,000 hours in 2 years to become a licensed professional counselor (LPC). Similar credentials include Licensed Mental Health Counselor (LMHC), Licensed Clinical Professional Counselor (LCPC), Licensed Professional Health Counselor of Mental Health (LPCC), Licensed Clinical Mental Health Counselor (LCMHC), Licensed Professional Counselor (LPC), etc. Similar requirements are applicable for other mental health professions including Marriage and Family Therapy, and Clinical Social Work.

In addition to becoming fully licensed the graduate may desire to obtain certification or specialization in a specific mental healthcare field to enhance career prospects. For example, the graduate may desire to obtain certification in Problematic Sexual Behavior Cognitive Behavioral Therapy (PSB-CBT), become a Registered Play Therapy (RPT), or become certified in Eye-movement Densitization and Reprocessing (EMDR), and/or the like. Each certification or specialization may have corresponding mentor qualification, and the graduate may be required to work under supervision of a mentor who has qualification corresponding to the field desired by the graduate. For example, a graduate desiring to become an RPT would be required to work under the supervision of a Registered Play Therapist Supervisor (RPT-S).

Identifying a mentor who may have the required qualification and availability to provide supervisory services may be challenging for the graduate or new professional. Typically, individual in this stage of their professional development rely on word-of-mouth or suggestions from friends, family, colleagues, etc., to identify qualified mentors who may be willing to provide supervisory services. This process of identifying mentors may be time-consuming, and may yield results that may not be satisfactory.

Furthermore, the mentors may have preferences for the types of mentees (or graduates) that the mentors may like to supervise. The mentors may be required to screen through multiple supervisory requests from the mentees, and manually shortlist the mentee(s) that the mentors may be willing to supervise based on mentors' preferences. This process of shortlisting the mentee(s) may be effort-intensive, and hence may cause inconvenience to the mentors.

Thus, there exists a need in the industry for a system and method that facilitate matchmaking between a mentee and a mentor.

It is with respect to these and other considerations that the disclosure made herein is presented.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments. Elements and/or components in the figures are not necessarily drawn to scale. Throughout this disclosure, depending on the context, singular and plural terminology may be used interchangeably.

FIG. 1 depicts an example environment in which techniques and structures for providing the systems and methods disclosed herein may be implemented.

FIG. 2 depicts a block diagram of an example user relationship management system in accordance with the present disclosure.

FIG. 3 depicts example snapshots of mentor and mentee user profile information in accordance with the present disclosure.

FIG. 4 depicts an example user interface depicting a mentor-mentee match, in accordance with the present disclosure.

FIG. 5 depicts a flow diagram of an example user relationship management method in accordance with the present disclosure.

### DETAILED DESCRIPTION

### Overview

The present disclosure describes a mentor-mentee relationship management system that facilitates match making between a mentor and a mentee. The system may be configured to receive mentor and mentee user profile and preference information, and determine a match between the mentor and the mentee by comparing respective mentor and mentee user profile and preference information. Specifically, the system may receive a request for mentor identification from the mentee including a target qualification that the mentee may aspire to obtain (or a qualification of a mentor that the mentee may be seeking). For example, the system may receive a request indicating that the mentee may be aspiring to become a Registered Play Therapist (RPT), and hence may seek a Registered Play Therapist Supervisor (RPT-S) for supervisory services. The system may identify a set of mentors who may have RPT-S as mentor qualification, and then compare mentee user profile and preference information with user profile and preference information of each identified mentor, and calculate match or similarity scores between the mentee and each mentor. The system may then identify one or more mentors having the match score greater than a predefined threshold as “matched” mentor(s) for the mentee.

In some aspects, the system may additionally determine whether the target qualification may be state-specific or generic to entire nation. If the system determines that the target qualification may be state-specific, the system may identify matching mentors for the mentee such that mentors may be located or have mentor qualification associated with the state corresponding to the target qualification. On the other hand, if the system determines that the target qualification may not be state-specific, the system may not restrict mentor identification to any specific state or jurisdiction.

In additional aspects, the system may calculate a risk score associated with the mentee based on mentee academic grades and/or mentee code of ethics infraction information, which may be part of mentee user profile information. The system may identify the mentor(s) for the mentee based on the calculated risk score and individual mentor preferences. For example, a mentor who may desire to provide supervisory services to only those mentees with low-risk scores, the system may match the mentor with the mentees having low risk scores.

In further aspects, the system may enable mentors to identify mentees to provide supervisory services. In addition, the system may enable mentors or mentees to identify peers for peer consultation. For example, if a mentor is a doctor handling a specific patient case, the system may enable the mentor to identify peers handling similar patient types, who may provide peer consultation to the mentor.

In additional aspects, the system enables the mentor and mentee to interact with each other via the system, schedule interview sessions or interactions, send reminders, etc. Further, the system may enable the mentor and the mentee to store or log mentor-mentee interaction (e.g., audio, video recordings of interactions, files, etc.) on the system. The mentor and the mentee may have their respective system logins, and the system may be Health Insurance Portability and Accountability Act (HIPPA) compliant.

The present disclosure discloses a mentor-mentee relationship management system that may facilitate a mentee (or a mentor) to conveniently and efficiently identify a qualified mentor (or a mentee) who may provide supervisory services to the mentee. Since the system uses mentor and mentee user profile and preference information to match the mentor and the mentee, the match making is accurate and the system provides relevant matches to the mentees/mentors that meet respective requirements or preferences. Further, the system provides a platform on which the mentors and mentees may store or log their interactions (e.g., for later retrieval and reference), thus enhancing user convenience.

These and other advantages of the present disclosure are provided in detail herein.

### Illustrative Embodiments

The disclosure will be described more fully hereinafter with reference to the accompanying drawings, in which example embodiments of the disclosure are shown, and not intended to be limiting.

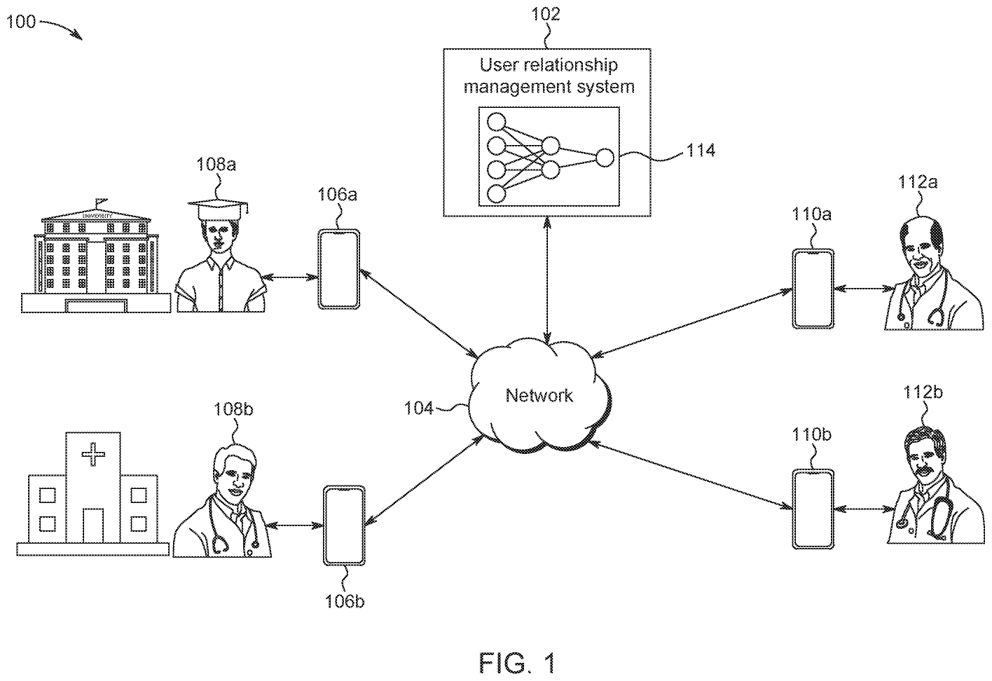

FIG. 1 depicts an example environment 100 in which techniques and structures for providing the systems and methods disclosed herein may be implemented. The environment 100 may include a user relationship management system 102 (or a system 102) connected with a plurality of user devices via one or more networks 104 (or a network 104). In some aspects, the system 102, as described herein, can be implemented in hardware, software (e.g., firmware), or a combination thereof.

The system 102 may be hosted on one or more servers (not shown) and may be an online platform that may facilitate a plurality of users to connect with each other via the network 104, and share and log user interactions on the platform. Specifically, the system 102 may facilitate one or more graduates or users (“mentees”) seeking mentorship or supervisory services to conveniently identify and connect with qualified mentors, based on mentor and mentee profiles and preferences. Similarly, the system 102 may facilitate the mentors to identify or receive supervisory requests from those mentees that meet mentors' requirements and/or preferences.

The network 104 may be, for example, a communication infrastructure in which the connected devices discussed in various embodiments of this disclosure may communicate. The network 104 may be and/or include the Internet, a private network, public network or other configuration that operates using any one or more known communication protocols such as, for example, transmission control protocol/Internet protocol (TCP/IP), Bluetooth®, BLE®, Wi-Fi based on the Institute of Electrical and Electronics Engineers (IEEE) standard 802.11, UWB, and cellular technologies such as Time Division Multiple Access (TDMA), Code Division Multiple Access (CDMA), High Speed Packet Access (HSPDA), Long-Term Evolution (LTE), Global System for Mobile Communications (GSM), and Fifth Generation (5G), to name a few examples.

The plurality of user devices may include, but is not limited to, mobile phones, laptops, computers, tablets, and other similar devices having communication capabilities. In some aspects, the plurality of user devices may be associated with a plurality of mentees and mentors who may access the system 102 to seek and provide supervisory services. For example, a mentee device 106a may be associated with a mentee 108a who may be a college graduate seeking supervisory services from a mentor. In an exemplary aspect, the mentee 108a may be a fresh out-of-college graduate of a mental healthcare program who may have cleared graduation exam, and hence may be a license-eligible user. As an example, the mentee 108a may access the system 102 (via the mentee device 106a) to identify a mentor who may provide supervisory services to the mentee 108a, so that the mentee 108a may become a licensed professional counselor (LPC). A person ordinarily skilled in the art may appreciate that that a license-eligible user may be required to work under supervision of a mentor or a supervisor for a predefined count of hours (e.g., 3,000 hours in 3 years) to become an LPC.

In addition, the mentee 108a may desire to obtain certification or specialization in a specific mental healthcare area/field, for which the mentee 108a may be required to work under supervision of a mentor who may be qualified to provide supervisory services in the specific mental healthcare area. For example, the mentee 108a may desire to become a Registered Play Therapist (RPT), for which the mentee 108a may be required to work under supervision of a Registered Play Therapist Supervisor (RPT-S). In this case, the mentee 108a may access the system 102 to identify an RPT-S who may be available to provide supervisory services to the mentee 108a.

In a similar manner, a mentee device 106b may be associated with a mentee 108b who may be an LPC desiring to obtain certification or specialization. For example, the mentee 108b may desire to obtain certification in Problematic Sexual Behavior Cognitive Behavioral Therapy (PSB-CBT), and may access the system 102 to identify qualified mentors who may be available to provide supervisory services to the mentee 108b. In other aspects, the mentee 108b may be a doctor who may desire to obtain peer consultation on a case that the mentee 108b may be handling. For example, the mentee 108b may be a hypnotherapist who may seek peer consultation on a patient case from an equally qualified peer (or a mentor with higher qualification) handling similar patient types. The mentee 108b may access the system 102 (via the mentee device 106b) to identify a qualified peer/mentor (in the same state where the mentee 108b may be located or in a different state/jurisdiction) who may provide consultation to the mentee 108b.

In further aspects, mentor devices 110a and 110b may be associated with mentors 112a and 112b respectively, who may be qualified supervisors eligible to provide supervisory services to the mentees 108a and 108b (and other mentees, not shown). The mentors 112a and 112b may access the system 102 (via the mentor devices 110a and 110b respectively) to receive supervisory requests from the mentees.

In some aspects, the system 102 may be configured to facilitate mentor-mentee match making, based on mentor and mentee profiles and preferences. The system 102 may be an Artificial Intelligence (AI)-based system that may include a neural network model 114. The neural network model 114 may be stored in a system 102 memory (not shown in FIG. 1). The neural network model 114 may be a trained or unsupervised neural network model that may analyze information included in the mentor and mentee profiles and preferences using machine learning and natural language processing, which may facilitate in mentor-mentee match making.

In one or more aspects, the neural network model 114 may include electronic data, which may be implemented, for example, as a software component, and may rely on code databases, libraries, scripts, or other logic or instructions for execution of a neural network algorithm by a system 102 processor (not shown in FIG. 1). The neural network model 114 may be implemented as code and routines configured to enable a computing device, such as the system 102, to perform one or more operations (such as perform mentor-mentee match making). In some aspects, the neural network model 114 may be implemented using hardware including a processor, a microprocessor (e.g., to perform mentor-mentee match making), a field-programmable gate array (FPGA), or an application-specific integrated circuit (ASIC). In other aspects, the neural network model 114 may be implemented by using a combination of hardware and software.

Examples of the neural network model 114 may include, but are not limited to, a deep neural network (DNN), a convolutional neural network (CNN), a recurrent neural network (RNN), a CNN-recurrent neural network (CNN-RNN), R-CNN, Fast R-CNN, Faster R-CNN, an artificial neural network (ANN), a Long Short Term Memory (LSTM) network based RNN, CNN+ANN, LSTM+ANN, a gated recurrent unit (GRU)-based RNN, a fully connected neural network, a deep Bayesian neural network, a Generative Adversarial Network (GAN), and/or a combination of such networks. In some aspects, the neural network model 114 may include numerical computation techniques using data flow graphs. In one or more aspects, the neural network model 114 may be based on a hybrid architecture of multiple Deep Neural Networks (DNNs).

In operation, the mentees 108a, 108b and the mentors 112a, 112b may create user accounts on the system 102 when the respective mentors and mentees access the system 102 for a first time, via respective user devices. The mentees 108a, 108b and the mentors 112a, 112b may provide information associated with respective user profiles and preferences while creating user accounts on the system 102. For example, the mentees 108a, 108b may provide user profile information to the system 102 which may include, but is not limited to, mentee name, mentee address, mentee availability to attend supervisory sessions with mentors, mentee college name, mentee academic grades, mentee experience (if any), and mentee code of ethics associated information. As an example, the mentee 108a may provide mentee availability as 15 hours per week for face-to-face interaction with the mentor and 10 hours per week for virtual or online interaction, mentee experience as zero or 1 year, mentee academic grade as 3.8 Grade Point Average (GPA), etc.

In some aspects, the mentee code of ethics associated information may include information corresponding to one or more code of ethics infractions that may be associated with the mentee 108a. In further aspects, the mentee preferences may include information associated with geolocation preference, gender preference, experience preference, and/or the like, associated with a mentor that the mentee 108b may desire to receive supervisory services from. In some aspects, the mentee preferences may be part of the user profile information that the mentee 108b may share with the system 102 while creating the user account, or may be provided separately by the mentee 108b after the system 102 creates the user account for the mentee 108b.

In a similar manner, the mentors 112a, 112b may provide user profile information to the system 102 which may include, but is not limited to, mentor name, mentor address, mentor availability to participate in face-to-face or virtual interactions with a mentee, mentor qualification information including mentor license type, mentor certification, mentor credential information, etc., mentor client type information, mentor experience, and mentor code of ethics associated information. As an example, the mentor 112a may input that the mentor 112a is a certified professional counselor supervisor (CPCS), an RPT-S, has 15 years of experience, and is based out of Texas, USA. The mentor preferences may include information associated with academic grades, code of ethics infraction information, certification or specialization information, and/or the like, associated with a mentee to whom the mentor 112a may desire to provide supervisory services. For example, the mentor 112a may have a preference that the mentor 112a may only provide supervisory services to a mentee who may have GPA of more than 3.6, and who may not have any code of ethics infractions.

Similar to the mentees 108a, 108b, the mentors 112a, 112b too may share the mentor preferences as part of the user profile information that the mentors 112a, 112b may share with the system 102 while creating respective user accounts or may share the mentor preferences separately after the system 102 creates the user accounts.

In some aspects, the mentees 108a, 108b and the mentors 112a, 112b may provide user profile information to the system 102 via respective user devices and the network 104. For example, the mentee 108a may input the user profile information on the mentee device 106a, and the mentee device 106a may transmit the information to the system 102 via the network 104.

Responsive to receiving the user profile information from the respective user devices, the system 102 may create user accounts for the mentees 108a, 108b and the mentors 112a, 112b. The mentees 108a, 108b and the mentors 112a, 112b may access the system 102 to seek and provide supervisory services when the system 102 creates the user accounts.

In an exemplary aspect, a mentee (e.g., the mentee 108a) may access the system 102, via the mentee device 106a, and send a request for mentor identification to the system 102 when a mentee 108a user account is created on the system 102. The request may include information associated with a license type or certification/specialization type for which the mentee 108a may be seeking supervisory services. For example, the mentee 108a may send the request indicating that the mentee 108a may desire to pursue certification in RPT, and hence may seek an RPT-S. In addition, the mentee 108a may provide additional mentee preferences (if any) associated with the mentor that the mentee 108a may desire to seek supervisory services from. For example, the mentee 108a may indicate in the request that the mentee 108a seeks a mentor who may be residing/located within 50 miles of mentee 108a address.

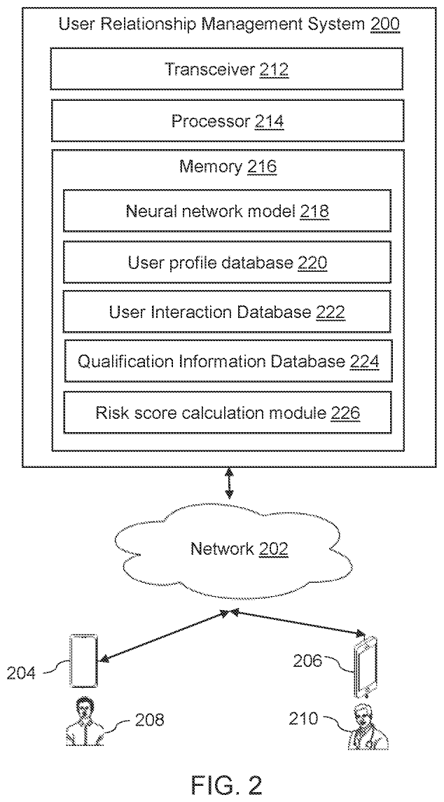

Responsive to receiving the request from the mentee 108a, the system 102 may use the neural network model 114 to analyze the information included in the request, mentee 108a user profile information, and user profile information for a plurality of mentors stored on the system 102, and shortlist/determine one or more mentors (e.g., the mentors 112a, 112b) who may be matched with the request. Specifically, the system 102 may use the neural network model 114 to identify a set of mentors who may be RPT-S from the plurality of mentors. Responsive to identifying the set of mentors, the neural network model 114 may compare user profile information of the mentee 108a and the set of mentors to calculate match scores between the mentee 108a user profile and user profile of each mentor from the set of mentors. Stated another way, the neural network model 114 may calculate match score between the mentee 108a with each identified mentor. The system 102 may then identify one or more mentors (e.g., the mentors 112a, 112b) who may be matched with the request based on the match scores. For example, the system 102 may identify those mentors to match with the request who may have respective match scores greater than a predefined threshold. The process of matching mentees with mentors is described later in conjunction with FIG. 2.

Responsive to determining the one or more mentors, the system 102 may transmit respective mentor profile information as “matched profile” to the mentee device 106a. Responsive to receiving the matched profile, the mentee 108a may view the profile and send a connect request to the corresponding mentor, via the system 102. The connect request may include a request to schedule an interview session with the mentor, so that mentor-mentee interaction may commence on the system 102.

Although the description above describes that the system 102 facilitates match making between mentors and mentees, the system 102 may further enable the mentors and the mentees to interact with each other via the system 102, and store or log mentor-mentee interactions (e.g., written comments, audio and/or video interactions) on the system 102 memory. The system 102 may be Health Insurance Portability and Accountability Act (HIPPA) compliant. The system 102 may additionally enable the mentors and the mentees to schedule interaction sessions, send or receive session reminders, log count of supervisory hours, and/or perform other actions that may facilitate mentor-mentee interaction.

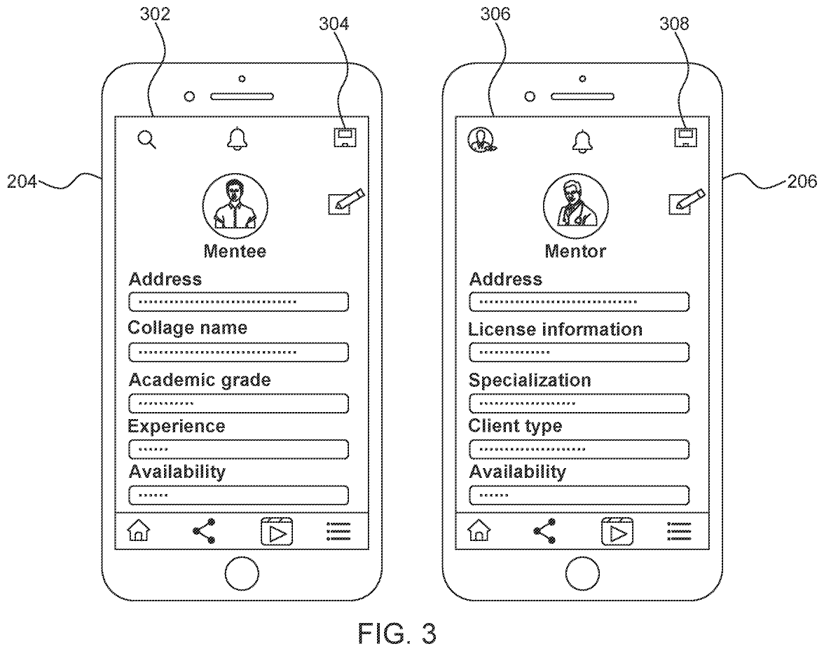

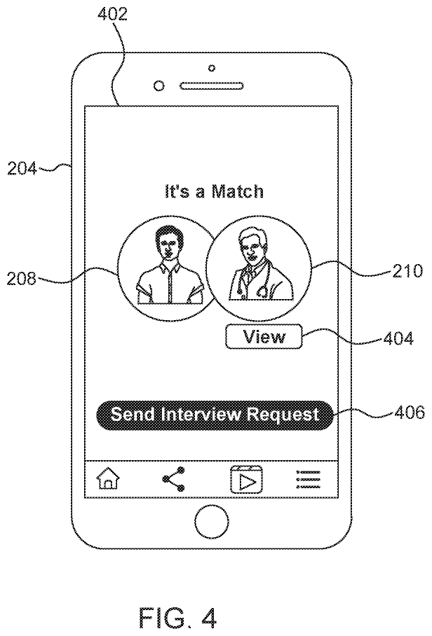

FIG. 2 depicts a block diagram of an example user relationship management system 200 (or a system 200) in accordance with the present disclosure. While describing FIG. 2, references may be made to FIGS. 3 and 4.

The system 200 may be same as the user relationship management system 102. The system 200 may be hosted on one or more servers (not shown) and may communicatively connect with a plurality of user devices via a network 202. For example, the system 200 may communicatively connect with a mentee device 204 and a mentor device 206, via the network 202. The mentee device 204 may be associated with a mentee 208 (who may be same as, for example, the mentee 108a or 108b), and the mentor device 206 may be associated with a mentor 210 (who may be same as, for example, the mentor 112a or 112b). The mentee device 204 may be same as the mentee device 106a, 106b, and the mentor device 206 may be same as the mentor device 110a, 110b. Further, the network 202 may be same as the network 104.

The system 200 may include a plurality of components including, but not limited to, a transceiver 212, a processor 214, and a memory 216, which may communicatively couple with each other via a bus (not shown).

In some aspects, the memory 216 may store programs in code and/or store data for performing various system 200 operations in accordance with the present disclosure. Specifically, the processor 214 may be configured and/or programmed to execute computer-executable instructions stored in the memory 216 for performing various system 200 functions in accordance with the disclosure. Consequently, the memory 216 may be used for storing code and/or data code and/or data for performing operations in accordance with the present disclosure.

In one or more aspects, the processor 214 may be disposed in communication with one or more memory devices (e.g., the memory 216 and/or one or more external databases (not shown in FIG. 2)). The memory 216 can include any one or a combination of volatile memory elements (e.g., dynamic random-access memory (DRAM), synchronous dynamic random-access memory (SDRAM), etc.) and can include any one or more nonvolatile memory elements (e.g., erasable programmable read-only memory (EPROM), flash memory, electronically erasable programmable read-only memory (EEPROM), programmable read-only memory (PROM), etc.).

The memory 216 may be one example of a non-transitory computer-readable medium and may be used to store programs in code and/or to store data for performing various operations in accordance with the disclosure. The instructions in the memory 216 can include one or more separate programs, each of which can include an ordered listing of computer-executable instructions for implementing logical functions. For example, the memory 216 may include a neural network model 218 (same as the neural network model 114) that may facilitate the system 200 in determining a match between a mentor and a mentee associated with the system 200.

In further aspects, the memory 216 may include a plurality of databases including, but not limited to, a user profile database 220, a user interaction database 222, and a qualification information database 224. The memory 216 may further include a risk score calculation module 226. The risk score calculation module 226, as described herein, may be stored in the form of computer-executable instructions, and the processor 214 may be configured and/or programmed to execute the stored computer-executable instructions for performing system 200 functions in accordance with the present disclosure. In one or more aspects, the processor 214 may use, via the neural network model 218 and the risk score calculation module 226, the information stored in the memory databases, to determine a match between a mentor and a mentee.

In operation, the transceiver 212 may be configured to receive user profile information from the mentee device 204 and the mentor device 206. As described in conjunction with FIG. 1, the mentee 208 and the mentor 210 may provide their respective user profile information to the system 200, when the mentee 208 and the mentor 210 access the system 200 for a first time. The mentee 208 and the mentor 210 may provide the user profile information to enable the system 200 to create user accounts for the mentee 208 and the mentor 210.

The mentee 208 and the mentor 210 may input respective user profile information on the mentee device 204 and the mentor device 206, and the respective devices may transmit the information to the transceiver 212 via the network 202. Examples of mentee 208 user profile information and mentor 210 user profile information are already described in conjunction with FIG. 1. Exemplary snapshots of user interfaces of the mentee device 204 and the mentor device 206 with respective user profile information is depicted in FIG. 3. Specifically, FIG. 3 depicts example snapshots of mentor and mentee user profile information in accordance with the present disclosure.

As shown in FIG. 3, the mentee 208 may input the mentee 208 user profile information on a user interface 302 of the mentee device 204. The mentee 208 user profile information may include mentee 208 photo, mentee 208 name, mentee 208 address, mentee 208 college name, mentee 208 experience (if any), mentee 208 availability, mentee 208 academic grade, and/or the like. The mentee 208 may click on a save or submit button 304 on the user interface 302, when the mentee 208 inputs the mentee 208 user profile information. Responsive to clicking the submit button 304, the mentee device 204 may transmit the mentee 208 user profile information to the transceiver 212.

In a similar manner, the mentor 210 may input the mentor 210 user profile information on a user interface 306 of the mentor device 206. The mentor 210 user profile information may include mentor 210 photo, mentor 210 name, mentor 210 address, mentor 210 qualification information including license type information and specialization/certification information, client type handled by the mentor 210, mentor 210 availability, and/or the like. The mentor 210 may click on a save or submit button 308 on the user interface 306, when the mentor 210 inputs the mentor 210 user profile information. Responsive to clicking the submit button 308, the mentor device 206 may transmit the mentor 210 user profile information to the transceiver 212.

As described in conjunction with FIG. 1, the mentee 208 and the mentor 210 may provide respective user preferences as part of the user profile information, or separately to the system 200. For example, the mentee 208 may provide preferences for a type of mentor that the mentee 208 may desire to seek supervisory services from. Similarly, the mentor 210 may provide preferences for a type of mentee to whom the mentor 210 may desire to provide supervisory services. For the description below, it is assumed that the mentee 208 and the mentor 210 provide respective user preferences as part of the user profile information.

Responsive to receiving the mentee 208 user profile information and the mentor 210 user profile information, the transceiver 212 may send the information to the user profile database 220 for storage purpose. In addition, the transceiver 212 may send the information to the processor 214. The processor 214 may use the information obtained from the transceiver 212 and create user accounts for the mentee 208 and the mentor 210 on the system 200.

In some aspects, the mentee 208 may transmit a request for mentor identification to the system 200 via the mentee device 204, when the system 200 creates mentee 208 user account. The request may include a target qualification for which for the mentee 208 may seek mentor's supervisory services. For example, the mentee 208 may input target qualification as certification or specialization that the mentee 208 may seek to obtain, e.g., Problematic Sexual Behavior Cognitive Behavioral Therapy (PSB-CBT) or Registered Play Therapy (RPT).

The transceiver 212 may receive the request, including the target qualification, from the mentee device 204 when the mentee 208 submits the request on the mentee device 204. Responsive to receiving the request, the transceiver 212 may send the request to the user profile database 220 for storage purpose.

Further, the processor 214 may obtain the request from the transceiver 212 (or the user profile database 220) when the mentee 208 submits the request on the mentee device 204. In addition, the processor 214 may obtain the mentee 208 user profile information and user profile information of a plurality of mentors (including the mentor 210) who may have user accounts on the system 200 from the user profile database 220. Responsive to obtaining the request, the mentee 208 user profile information and the user profile information for the plurality of mentors, the processor 214 may use the neural network model 218 to identify one or more mentors who may provide supervisory services to the mentee 208. Specifically, the processor 214 may use the neural network model 218 to execute one or more mentor-mentee match identification algorithms separately or in combination with each other to identify one or more mentors for the mentee 208. The different algorithms or approaches used by the processor 214 to identify one or more mentors for the mentee 208 are described below. Although the algorithms/approaches are described sequentially below, the order of description of the algorithms/approaches should not be construed as the order of executing the algorithms. The processor 214 may execute the algorithms/approaches in a different order or may execute the algorithms/approaches in parallel with each other.

The processor 214 may identify (via the neural network model 218) a set of mentors (e.g., including the mentor 210), from the plurality of mentors, who may be qualified to provide supervisory services to the mentee 208 based on the target qualification included in the request. Specifically, the processor 214 may fetch a first mapping of qualifications with respective mentor qualification nomenclature from the qualification information database 224, in response to obtaining the request from the transceiver 212. In some aspects, the qualification information database 224 may pre-store the first mapping of qualifications with the respective mentor qualification nomenclature, and the processor 214 may use the first mapping to identify a mentor qualification nomenclature corresponding to the target qualification included in the request. For example, if the target qualification included in the request is RPT, the processor 214 may determine from the first mapping that the corresponding mentor qualification nomenclature may include RPT-S.

Responsive to determining the mentor qualification nomenclature, the processor 214 may use the neural network model 218 to identify the set of mentors, from the plurality of mentors, who may be qualified as RPT-S. Specifically, the neural network model 218 may parse the user profile information of the plurality of mentors, and shortlist the set of mentors having certification or specialization as RPT-S.

In addition to identifying the set of mentors based on the first mapping of qualifications with the mentor qualification nomenclature (as described above), in some aspects, the processor 214 may execute an additional step or perform an additional algorithm to identify the set of mentors. For example, responsive to receiving the target qualification included in the request, the processor 214 may fetch a second mapping of qualification with a plurality of geographical parameters from the qualification information database 224. In some aspects, the qualification information database 224 may pre-store the second mapping of qualifications with the plurality of geographical parameters, and the processor 214 may use the second mapping to identify the set of mentors from the plurality of mentors. In an exemplary aspect, a geographical parameter associated with a qualification may indicate whether the qualification may be governed by state laws/rules or Federal laws/rules. Stated another way, a geographical parameter associated with a qualification may indicate whether the qualification is state-specific (and hence may require a mentor who may be located or qualified in the state to provide supervisory services to the mentee 208), or generic to entire nation.

Responsive to fetching the second mapping, the processor 214 may determine whether the target qualification included in the request is specific to a state (e.g., a state where the mentee 208 may be located) or national. If the processor 214 determines that the target qualification is state-specific, the processor 214 may further determine a corresponding state name (e.g., Texas) from the second mapping, and identify the set of mentors, from the plurality of mentors, who may be in the corresponding state (e.g., Texas). In this case, the processor 214 may determine state information for the plurality of mentors from mentor addresses that may be included in mentor user profile information stored in the user profile database 220.

On the other hand, if the processor 214 determines that the target qualification is not state-specific, the processor 214 may not restrict identification of the set of mentors to any specific state or jurisdiction.

In further aspects, if the mentee 208 user preferences include geolocation distance preference of a mentor that the mentee 208 may be seeking, the processor 214 may identify the set of mentors based on the geolocation distance preference. For example, if the mentee 208 prefers to identify a mentor who may be located within 50 miles of the mentee 208 address, the processor 214 may identify the set of mentors such that the address of each mentor from the set of mentors may be located within 50 miles of the mentee 208 address. In this case, the processor 214 may fetch the mentor addresses for the plurality of mentors and calculate a geo-distance between the address of each mentor and the mentee 208 address. The processor 214 may then compare the calculated geo-distances with the geolocation distance preference (e.g., 50 miles), and identify the set of mentors who may have the calculated geo-distances less than the geolocation distance preference.

Although the description above describes that the mentee 208 user preferences include the geolocation distance preference, in some aspects, the mentee 208 may also transmit, via the mentee device 204, the geolocation preference as part of the request for mentor identification.

Responsive to identifying the set of mentors, the processor 214 may use the neural network model 218 to identify one or more mentors, from the identified set of mentors, who may be identified as a match (e.g., a mentee-mentor match) for the mentee 208. In some aspects, the processor 214 (via the neural network model 218) may compare the mentee 208 user profile information with the user profile information of each mentor from the set of mentors and calculate match scores between mentee 208 user profile and each mentor user profile from the set of mentors. For example, the neural network model 218 may compare the mentee 208 user profile information with the mentor 210 user profile information and calculate a match score between the mentee 208 user profile and mentor 210 user profile based on the comparison.

In some aspects, the processor 214 may determine the match score between the mentee 208 user profile and the mentor 210 user profile by calculating similarity scores for each user profile information parameter for the mentee 208 and the mentor 210. For example, the processor 214 may compare mentee 208 and mentor 210 addresses and determine a location proximity or similarity score. In some aspects, the location similarity score may be high (e.g., 8 or 9 on a scale of 10), when a geolocation distance between mentee 208 and mentor 210 addresses is small (e.g., less than 30 miles), and may be low (e.g., 2 or 3) when the geolocation distance is large (e.g., more than 500 miles).

In a similar manner, the processor 214 may calculate an academic grade similarity score by calculating a difference between mentee 208 academic grades and mentor 210 preferred academic grades for a mentee that the mentor 210 may prefer to provide supervisory services (that may be part of mentor 210 user preferences included in the mentor 210 user profile information). In some aspects, the academic grade similarity score may be high (e.g., 8 or 9) when the difference is less than a threshold (e.g., 0.1 GPA) and may be low (e.g., 2 or 3) when the difference is large (e.g., more than 0.5 GPA).

As another example, the processor 214 may calculate a code of ethics infraction score based on infractions included in the mentee 208 user profile information and mentor 210 preferred infractions for the mentee that the mentor 210 may prefer to provide supervisory services. In some aspects, the code of ethics infraction score may be low (e.g., 2 or 3) when the mentee 208 user profile information indicates one or more infractions and the mentor 210 prefers a mentee with no infractions.

In a similar manner, the processor 214 may compare parameters such as availability, gender, experience, etc., included in the mentee 208 user profile information and the mentor 210 user profile information to calculate individual similarity score for each parameter.

Responsive to calculating the similarity scores, the processor 214 may perform a weighted summation of the individual similarity scores to calculate the match score between the mentee 208 user profile and the mentor 210 user profile. For example, response to calculating the similarity scores, the processor 214 may fetch weights associated with each parameter from the memory 216 (that may pre-store the corresponding weights) and use the weights to calculate the weighted sum of the individual similarity scores. The weighted sum may be the match score between the mentee 208 user profile and the mentor 210 user profile.

In a similar manner, the processor 214 may calculate match scores between the mentee 208 user profile and user profile of each mentor from the set of mentors. Responsive to calculating the match scores, the processor 214 may compare the calculated match scores with a predefined threshold (which may be, for example, 8). The processor 214 may identify one or more mentors who may have the match scores greater than the threshold as “matches” for the mentee 208. For example, the processor 214 may identify the mentor 210 as a match for the mentee 208 if the match score for the mentor 210 is greater than the predefined threshold (e.g., 8).

A person ordinarily skilled in the art may appreciate that since the processor 214 uses user profile information (that may include user preferences) of the mentees and mentors, the processor 214 identifies the match that may be based on specific mentee and mentor preferences. Therefore, if a mentor prefers to provide supervisory services to a mentee with GPA greater than 3.6, the processor 214 may identify a “matched” mentee for the mentor such that the mentee may have a GPA of more than 3.6. This may enhance mentee and mentor convenience, as the processor 214 identifies matches based on individual preferences.

In further aspects, in addition to identifying the one or more mentors based on match scores (as described above), the processor 214 may identify the one or more mentors based on a “risk score” associated with the mentee 208. Specifically, the processor 214 may use the instructions stored in the risk score calculation module 226 and calculate a risk score associated with the mentee 208 based on one or more parameters including, but not limited to, mentee 208 academic grades and mentee 208 code of ethics infraction information. For example, the processor 214 may calculate a high-risk score (e.g., 8 or 9) for the mentee 208 if the mentee 208 academic grades are low (e.g., less than 2.5 GPA) and the mentee 208 user profile indicates one or more code of ethics infractions. On the other hand, the processor 214 may calculate a low-risk score (e.g., 2 or 3) if the mentee 208 academic grades are above average (e.g., more than 3.5 GPA) and the mentee 208 user profile does not indicate any code of ethics infraction.

Responsive to calculating the risk score, the processor 214 may compare the calculated risk score with a risk threshold value (e.g., 6), and determine whether the calculated risk score is less than or more than the risk threshold value. In some aspects, the risk threshold value may be part of mentor user preferences and indicate a mentor preference with respect to the “risk” associated with the mentee that the mentor may be willing to provide supervisory services. For example, the mentor 210 user preferences may indicate that the mentor 210 may only provide supervisory services to a mentee who may have corresponding risk score of less than 6. In this case, while identifying the one or more mentors for the mentee 208, the processor 214 may determine whether the mentee 208 has the calculated risk score of more than or less than 6. If the calculated risk score is more than 6, the mentor 210 may not be identified as a “matched” mentor for the mentee 208. On the other hand, if the calculated risk score is less than 6, the mentor 210 may be identified as the matched mentor for the mentee 208.

Responsive to identifying the one or more mentors (e.g., the mentor 210) as the matched mentor(s) for the mentee 208 based on the algorithms/approaches described above, the processor 214 may transmit, via the transceiver 212, mentor user profiles (e.g., the mentor 210 user profile) to the mentee device 204. The mentee 208 may view the mentor profile(s) on mentee device 204 user interface. An exemplary user interface depicting a matched mentor profile is shown in FIG. 4. Specifically, FIG. 4 depicts an example user interface 402 depicting a mentor-mentee match, in accordance with the present disclosure.

The user interface 402 may be the mentee device 204 user interface and may display a mentee 208 match with the mentor 210. The mentee 208 may click on a “View” button 404 to view the mentor 210 profile. Further, the mentee 208 may click on a “Send Interview Request” button 406 to schedule an interview session with the mentor 210. In some aspects, the mentee 208 and the mentor 210 may enter a mentor-mentee relationship post the interview session.

The system 200 may configured to store/log interactions between the mentor 210 and the mentee 208 on the user interaction database 222. For example, the user interaction database 222 may store audio or video interactions between the mentor 210 and the mentee 208, written comments, notes or messages exchanged between the mentor 210 and the mentee 208 via system 200, etc. In addition, the system 200 may enable the mentor 210 and the mentee 208 to schedule interaction sessions. Furthermore, the system 200 may store a count of hours that the mentor 210 and the mentee 208 spend on interactions and may store the count of hours in the user profile database 220. The mentee 208 and/or the mentor 210 may view the count of hours by accessing respective user profiles.

Further, the system 200 may enable the mentee 208 or the mentor 210 to end the mentee-mentor relationship after the mentor-mentee relationship is established. For example, the mentee 208 or the mentor 210 may transmit, via their respective devices, a relationship termination request to the system 200 when the mentee 208 or the mentor 210 desires to terminate the relationship. Responsive to receiving the request, the system 200 may disable communication or interaction between the mentee 208 and the mentor 210.

Although FIG. 4 depicts the mentee 208 match with a single mentor (e.g., the mentor 210), in some aspects, the processor 214 may transmit more than one mentor profile to the mentee device 204. In this case, the mentor profiles may be displayed as a “sorted” list, based on the calculated match scores. For example, a mentor with highest match score may be displayed at the top position in the list, and a mentor with lowest match score may be displayed at the last position in the list.

Furthermore, although the description above describes an aspect where a mentee (e.g., the mentee 208) sends the request for mentor identification to the system 200, the present disclosure is not limited to the aspect of the mentee sending the request to the system 200. For example, the mentor 210 may also send a request for mentee identification to the system 200, and the system 200 may identify one or more matched mentees for the mentor 210 in a similar manner as described above. In addition, a mentor may also seek peer consultation on the system 200, by sending a request for peer identification. In this case also, the system 200 may identify one or more matched peers for the mentor in the similar manner as described above.

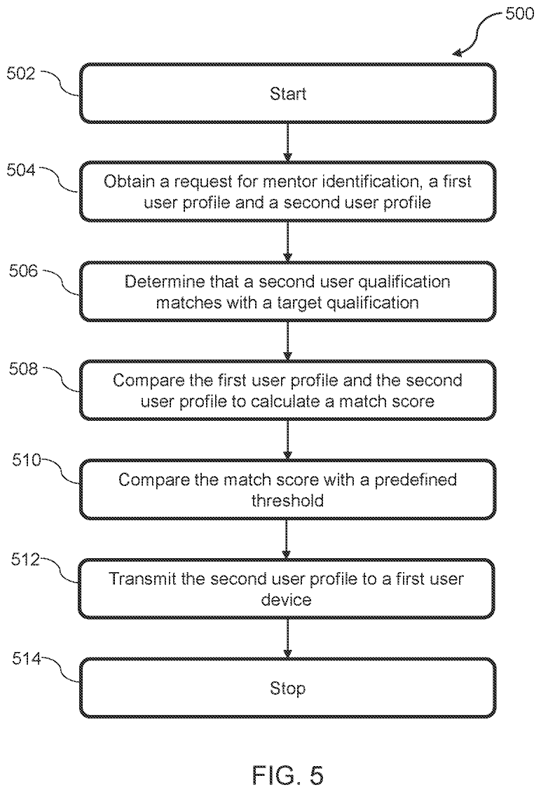

FIG. 5 depicts a flow diagram of an example user relationship management method 500 in accordance with the present disclosure. FIG. 5 may be described with continued reference to prior figures, including FIGS. 1-4. The following process is exemplary and not confined to the steps described hereafter. Moreover, alternative embodiments may include more or less steps that are shown or described herein and may include these steps in a different order than the order described in the following example embodiments.

Referring to FIG. 5, at step 502, the method 500 may commence. At step 504, the method 500 may include obtaining, by the processor 214, the request for mentor identification, the mentee 208 user profile information and the mentor 210 user profile information from the transceiver 212. As described in conjunction with FIG. 2, the request for mentor identification may include a target qualification for which for the mentee 208 may seek mentor's supervisory services.

At step 506, the method 500 may include determining, by the processor 214, that mentor 210 qualification matches with the target qualification. At step 508, the method 500 may include comparing, by the processor 214, the mentee 208 user profile and the mentor 210 user profile to calculate a match score between the mentee 208 user profile and the mentor 210 user profile in response to determining that the mentor 210 qualification matches with the target qualification. The process of calculating the match score is already described in conjunction with FIG. 2.

At step 510, the method 500 may include comparing, by the processor 214, the match score with a predefined threshold. At step 512, the method 500 may include transmitting, by the processor 214 (via the transceiver 212), the mentor 210 user profile to the mentee device 204 when the match score is greater than the predefined threshold.

At step 514, the method 500 may stop.

In the above disclosure, reference has been made to the accompanying drawings, which form a part hereof, which illustrate specific implementations in which the present disclosure may be practiced. It is understood that other implementations may be utilized, and structural changes may be made without departing from the scope of the present disclosure. References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a feature, structure, or characteristic is described in connection with an embodiment, one skilled in the art will recognize such feature, structure, or characteristic in connection with other embodiments whether or not explicitly described.

Further, where appropriate, the functions described herein can be performed in one or more of hardware, software, firmware, digital components, or analog components. For example, one or more application specific integrated circuits (ASICs) can be programmed to carry out one or more of the systems and procedures described herein. Certain terms are used throughout the description and claims refer to particular system components. As one skilled in the art will appreciate, components may be referred to by different names. This document does not intend to distinguish between components that differ in name, but not function.

It should also be understood that the word “example” as used herein is intended to be non-exclusionary and non-limiting in nature. More particularly, the word “example” as used herein indicates one among several examples, and it should be understood that no undue emphasis or preference is being directed to the particular example being described.

A computer-readable medium (also referred to as a processor-readable medium) includes any non-transitory (e.g., tangible) medium that participates in providing data (e.g., instructions) that may be read by a computer (e.g., by a processor of a computer). Such a medium may take many forms, including, but not limited to, non-volatile media and volatile media. Computing devices may include computer-executable instructions, where the instructions may be executable by one or more computing devices such as those listed above and stored on a computer-readable medium.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating various embodiments and should in no way be construed so as to limit the claims.

Accordingly, it is to be understood that the above description is intended to be illustrative and not restrictive. Many embodiments and applications other than the examples provided would be apparent upon reading the above description. The scope should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

All terms used in the claims are intended to be given their ordinary meanings as understood by those knowledgeable in the technologies described herein unless an explicit indication to the contrary is made herein. In particular, use of the singular articles such as “a,” “the,” “said,” etc. should be read to recite one or more of the indicated elements unless a claim recites an explicit limitation to the contrary. Conditional language, such as, among others, “can,” “could,” “might,” or “may,” unless specifically stated otherwise, or otherwise understood within the context as used, is generally intended to convey that certain embodiments could include, while other embodiments may not include, certain features, elements, and/or steps. Thus, such conditional language is not generally intended to imply that features, elements, and/or steps are in any way required for one or more embodiments.

That which is claimed is:

1. A user relationship management system comprising:
a transceiver configured to:
receive a first user profile from a first user device, and a second user profile from a second user device, wherein the second user profile comprises a second user qualification;
receive a target qualification from the first user device;
a processor communicatively coupled to the transceiver, wherein the processor is configured to:
obtain the first user profile, the second user profile, and the target qualification from the transceiver;
determine that the second user qualification matches with the target qualification;
compare the first user profile and the second user profile to calculate a match score between the first user profile and the second user profile responsive to a determination that the second user qualification matches with the target qualification;
compare the match score with a predefined threshold; and
transmit the second user profile to the first user device when the match score is greater than the predefined threshold.

2. The user relationship management system of claim 1, wherein the first user profile comprises at least one of: a first user name, a first user address, first user availability information, a first user college name, first user academic grades, first user experience information, first user preferences, and first user code of ethics associated information.

3. The user relationship management system of claim 2, wherein the second user profile comprises at least one of: a second user name, a second user address, second user availability information, second user license type information, second user certification information, second user client type information, second user mentor associated credential information, second user preferences, second user experience information, and second user code of ethics associated information.

4. The user relationship management system of claim 3 further comprising a memory configured to store a mapping of a plurality of geographical parameters with a plurality of qualifications.

5. The user relationship management system of claim 4, wherein the processor is further configured to:
fetch the mapping from the memory; and
determine a geographical parameter associated with the target qualification based on the mapping.

6. The user relationship management system of claim 5, wherein the processor is further configured to:
determine a geographic area associated with the target qualification based on the geographical parameter; and
determine that the second user address is located in the geographical area,
wherein the processor transmits the second user profile to the first user device when the second user address is located in the geographical area.

7. The user relationship management system of claim 3, wherein the transceiver is further configured to receive a geo-distance threshold between the first user address and the second user address from the first user device.

8. The user relationship management system of claim 7, wherein the processor is further configured to determine that a geo-distance between the first user address and the second user address is less than the geo-distance threshold, and wherein the processor transmits the second user profile to the first user device when the geo-distance is less than the geo-distance threshold.

9. The user relationship management system of claim 3, wherein the processor is further configured to calculate a first user risk score based on at least one of: the first user code of ethics associated information and the first user academic grades.

10. The user relationship management system of claim 9, wherein the processor is further configured to:
compare the first user risk score with a risk score threshold, wherein the second user preferences comprise the risk score threshold; and
determine that the first user risk score is less than the risk score threshold,
wherein the processor transmits the second user profile to the first user device when the first user risk score is less than the risk score threshold.

11. A user relationship management method comprising:
obtaining, by a processor, a first user profile, a second user profile, and a target qualification from a transceiver, wherein the transceiver is configured to:
receive the first user profile from a first user device and the second user profile from a second user device, wherein the second user profile comprises a second user qualification,
receive the target qualification from the first user device;
determining, by the processor, that the second user qualification matches with the target qualification;
comparing, by the processor, the first user profile and the second user profile to calculate a match score between the first user profile and the second user profile responsive to determining that the second user qualification matches with the target qualification;
comparing, by the processor, the match score with a predefined threshold; and
transmitting, by the processor, the second user profile to the first user device when the match score is greater than the predefined threshold.

12. The user relationship management method of claim 11, wherein the first user profile comprises at least one of: a first user name, a first user address, first user availability information, a first user college name, first user academic grades, first user experience information, first user preferences, and first user code of ethics associated information.

13. The user relationship management method of claim 12, wherein the second user profile comprises at least one of: a second user name, a second user address, second user availability information, second user license type information, second user certification information, second user client type information, second user mentor associated credential information, second user preferences, second user experience information, and second user code of ethics associated information.

14. The user relationship management method of claim 13 further comprising storing a mapping of a plurality of geographical parameters with a plurality of qualifications in a memory.

15. The user relationship management method of claim 14 further comprising:
fetching the mapping from the memory; and
determining a geographical parameter associated with the target qualification based on the mapping.

16. The user relationship management method of claim 15 further comprising:
determining a geographic area associated with the target qualification based on the geographical parameter; and
determining that the second user address is located in the geographical area,
wherein transmitting the second user profile to the first user device comprises transmitting the second user profile when the second user address is located in the geographical area.

17. The user relationship management method of claim 13 further comprising receiving a geo-distance threshold between the first user address and the second user address.

18. The user relationship management method of claim 17 further comprising determining that a geo-distance between the first user address and the second user address is less than the geo-distance threshold, wherein transmitting the second user profile to the first user device comprises transmitting the second user profile when the geo-distance is less than the geo-distance threshold.

19. The user relationship management method of claim 13 further comprising:
calculating a first user risk score based on at least one of: the first user code of ethics associated information and the first user academic grades;
comparing the first user risk score with a risk score threshold, wherein the second user preferences comprise the risk score threshold; and
determining that the first user risk score is less than the risk score threshold,
wherein transmitting the second user profile to the first user device comprises transmitting the second user profile when the first user risk score is less than the risk score threshold.

20. A non-transitory computer-readable storage medium in a distributed computing system, the non-transitory computer-readable storage medium having instructions stored thereupon which, when executed by a processor, cause the processor to:
obtain a first user profile, a second user profile, and a target qualification from a transceiver, wherein the transceiver is configured to:
receive the first user profile from a first user device and the second user profile from a second user device, wherein the second user profile comprises a second user qualification,
receive the target qualification from the first user device;
determine that the second user qualification matches with the target qualification;
compare the first user profile and the second user profile to calculate a match score between the first user profile and the second user profile responsive to determining that the second user qualification matches with the target qualification;
compare the match score with a predefined threshold; and
transmit the second user profile to the first user device when the match score is greater than the predefined threshold.
